(19) United States
(12) Patent Application Publication
(10) Pub. No.: US 2014/0074247 A1
(43) Pub. Date: Mar. 13, 2014

(54) JOINT RECONSTRUCTION SYSTEM AND METHOD

(71) Applicants: Kevin L. Ohashi, Jamaica Plain, MA (US); Donald E. Running, Warsaw, IN (US); Robert J. Ball, West Olive, MI (US); Randip R. Bindra, Clarendon Hills, IL (US); John J. Fernandez, Chicago, IL (US); Dale R. Peterson, Carlsbad, CA (US)

(72) Inventors: Kevin L. Ohashi, Jamaica Plain, MA (US); Donald E. Running, Warsaw, IN (US); Robert J. Ball, West Olive, MI (US); Randip R. Bindra, Clarendon Hills, IL (US); John J. Fernandez, Chicago, IL (US); Dale R. Peterson, Carlsbad, CA (US)

(21) Appl. No.: 13/319,068

(22) PCT Filed: May 7, 2010
(86) PCT No.: PCT/US10/34063

§ 371 Date: Mar. 4, 2013

Related U.S. Application Data
(60) Provisional application No. 61/176,529, filed on May 8, 2009.

(51) Int. Cl. A61F 2/46; A61F 2/42

(52) U.S. Cl. CPC A61F 2/4601; A61F 2/4261
USPC 623/21.12

(57) ABSTRACT

The implant (20) is shaped to generally mimic the form of the trapezium (12), or portions thereof. For example, the implant optionally defines a first metacarpal projection (32) and a second metacarpal projection (34) spaced from the first metacarpal projection to form a C-type, or cuff-type receptacle (36) for receiving the first metacarpal (15) such as the first and second metacarpal projections extend along either side of the first metacarpal. The receptacle optionally acts as a support surface (36A) for the first metacarpal during articulation thereof. In some embodiments, the first metacarpal projection is configured to extend adjacent the end portion of the first metacarpal that is adjacent the CMC joint (10) and the second metacarpal projection is configured to extend into the space (19) between the end portions of the first and second metacarpals (15, 18), thereby helping maintain the intermetacarpal spacing between the first and second metacarpals. The implant may comprise an inflatable cover for minimally invasive implantation.

### RELATED APPLICATION AND PRIORITY CLAIM

This application claims priority to U.S. Provisional Patent Application 61/176,529, filed on May 8, 2009, and entitled “TRAPEZIUM RECONSTRUCTION SYSTEM AND METHOD,” the contents of which are incorporated herein by reference in their entirety.

### TECHNICAL FIELD

The present invention is related to the field of carpometacarpal implants. In particular, the present invention relates to fixed and inflatable carpometacarpal implants.

### BACKGROUND

The carpometacarpal (CMC) joint of the thumb is also known as the trapeziometacarpal joint (TMC) because it connects the trapezium to the first metacarpal bone, the metacarpal bone of the thumb. Osteoarthritis of the CMC is severely painful and disabling, as the CMC is one of the most significant joints between the wrist and the metacarpus. Clinically, excisional arthroplasty of the trapezium is associated with pain as a result of osteoarthritic changes or changes due to disease of the joint surfaces between the metacarpal-trapezium, scaphoid-trapezium, and/or trapezoid-trapezium. During stage one or stage two arthritis of the carpometacarpal (CMC) joint, surgery is generally needed and the trapezium is typically removed. However, removal of the trapezium can result in proximal and radial migration of the metacarpal, leading to decreased grip strength and pinch strength.

While some methods for treating arthritis of the CMC joint have been proposed, generally, conventional carpometacarpal implants do not address subluxation or stabilization of the joint.

One method of treating arthritis of the CMC joint is ligament reconstruction and tendon interposition (LRTI). In a LRTI procedure, typically half of the flexor carpi radialis is harvested and suspensionplasty and ligament interposition performed. Alternatively, fascia lata or other tendon/ligament allografts or autografts can be used. However, this may lengthen the operating time due to harvest time and may increase morbidity and scarring.

A second method of treating arthritis of the CMC joint is by the use of silicone implants. While silicone implants can be effective, there may be problems with material wear, cold flow and foreign body synovitis which leads to destructive bone osteolysis, and loosening of the implant.

Lastly, allograft or xenograft materials have also been used to treat arthritis of the CMC joint. Allografts such as xenograft patches, “GORE-TEX” grafts, polypropylene implants and polyurethane implants have been used to resurface the carpometacarpal. However, allograft or xenograft materials do not stabilize the CMC joint because the materials are generally not shaped to support loads at the joint. In addition, there are concerns with potential occurrences of biological reactions with these materials.

### SUMMARY

Some aspects relate to an implant for a joint including a first bone having a first end portion and a second bone having a second end portion, the first and second end portions extending next to one another and being spaced from one another. The implant includes a body formed of resilient material. The body includes a first projection adapted to extend into a space between the first and second end portions of the first and second bones and a receptacle for receiving the first end portion of the first bone.

Some aspects relate to a method of delivering an implant into a joint following excision of a bone from the joint. The method includes excising at least a portion of a bone from a joint to leave a bone space, where a first end portion of a first bone extends next to a second end portion of a second bone, a first end portion of the first bone being spaced from a second end portion of the second bone. The bone space is accessed with a delivery device maintaining a cover of an implant, the cover being collapsed such that the implant is in a first, substantially compact state. The cover of the implant is positioned at a desired location in the bone space. Material is injected through the delivery device into the cover to transition the implant to a second, expanded state that is of substantially greater size that the first, compact state, such that at least a portion of the implant extends into the space between the first and second end portions of the first and second bones, respectively. The injectable material is allowed to set to form a substantially resilient body of the implant.

Some aspects relate to a carpometacarpal (CMC) implant that supports the first metacarpal, acting as a spacer. In some embodiments, the CMC implant is pre-shaped and/or inflatable. The CMC implant is optionally substantially flexible and adapted to move with the first CMC joint while being resilient enough to resist compression under natural loading of the first CMC joint.

In some embodiments, a carpometacarpal (CMC) implant for a trapezial space left by a trapezium excision includes a body formed of resilient material, where the body includes a metacarpal projection adapted to extend into an intermetacarpal space between the first metacarpal and a second metacarpal of the hand that is adjacent to the first metacarpal.

Other aspects relate to methods of treating a first CMC joint with a CMC implant. In some embodiments, a method of delivering a carpometacarpal (CMC) implant into a trapezial space includes excising at least a portion of a trapezium of a first carpometacarpal joint of a hand to define a trapezial space. The trapezial space is percutaneously accessed with delivery device maintaining a cover of a CMC implant, the cover being in a first, substantially compact state. The cover is positioned at a desired location in the trapezial space. Material is injected through the delivery device into the cover to transition the cover, and thus the implant, to a second, expanded state that is of substantially greater size that the first, compact state. The injectable material is then allowed to set to form a substantially resilient body of the CMC implant.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a schematic view of five carpometacarpal (CMC) joints of a hand, with a trapezium of a first of the CMC joints removed.

FIG. 2 illustrates a first CMC implant, according to some embodiments.

FIGS. 3 and 4 illustrate an expandable version of the first CMC implant of FIG. 2, according to some embodiments.

FIG. 5 illustrates a second CMC implant, according to some embodiments.

FIG. 6 illustrates a third CMC implant, according to some embodiments.

FIG. 7 illustrates a fourth CMC implant, according to some embodiments.

FIG. 8 illustrates a fifth CMC implant, according to some embodiments.

While the invention is amenable to various modifications, permutations, and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

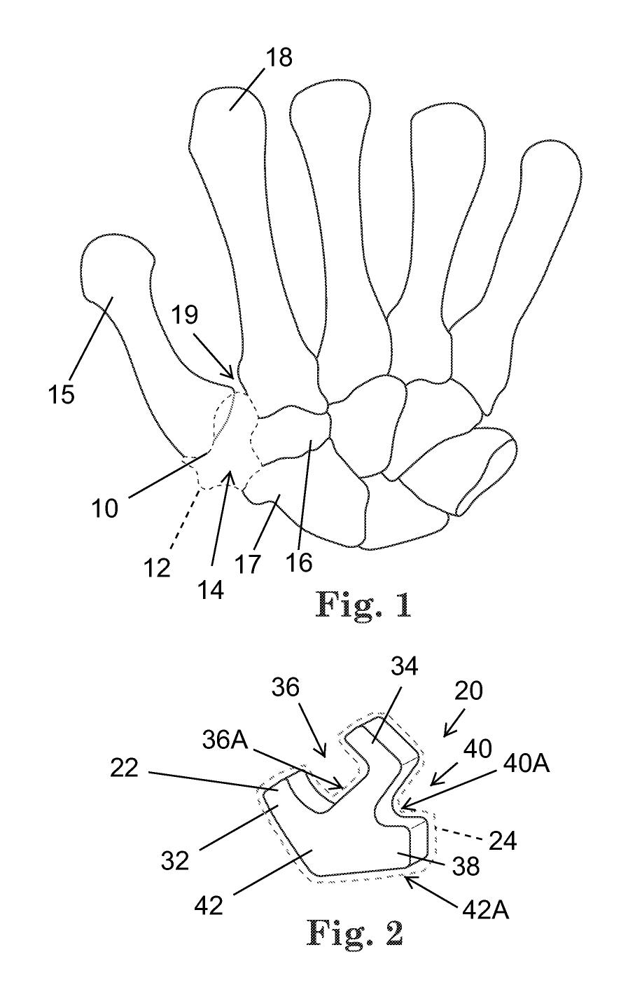

FIG. 1 shows a schematic view of a first carpometacarpal (CMC) joint 10 with a trapezium 12 naturally forming part of the CMC joint 10 removed thereby leaving a trapezial space 14 as indicated in broken lines. As shown, the CMC joint 10 is formed by the trapezium 12 and a first metacarpal 15. A trapezoid 16 and scaphoid 17 are positioned adjacent the trapezial space 14 and a second metacarpal 18 is located adjacent to the first metacarpal 15, where end portions of the first and second metacarpals naturally define a space 19 between them. Although, the first and second metacarpals 15, 18 are able to be articulated through a variety of relative angular positions, throughout articulation the end portions of the first and second metacarpals 15, 18 extend adjacent to one another, or side-by-side one another.

FIG. 2 shows a CMC implant 20 used to restore and/or stabilize subluxation of the trapezial space 14 following partial or total excision of the trapezium 12, according to some embodiments. Although the implant 20 is described in association with partial or total excision of the trapezium 12, in other embodiments the implant 20 is sized and shaped to be inserted into the interface between the first metacarpal 15 and the trapezium 12, for example.

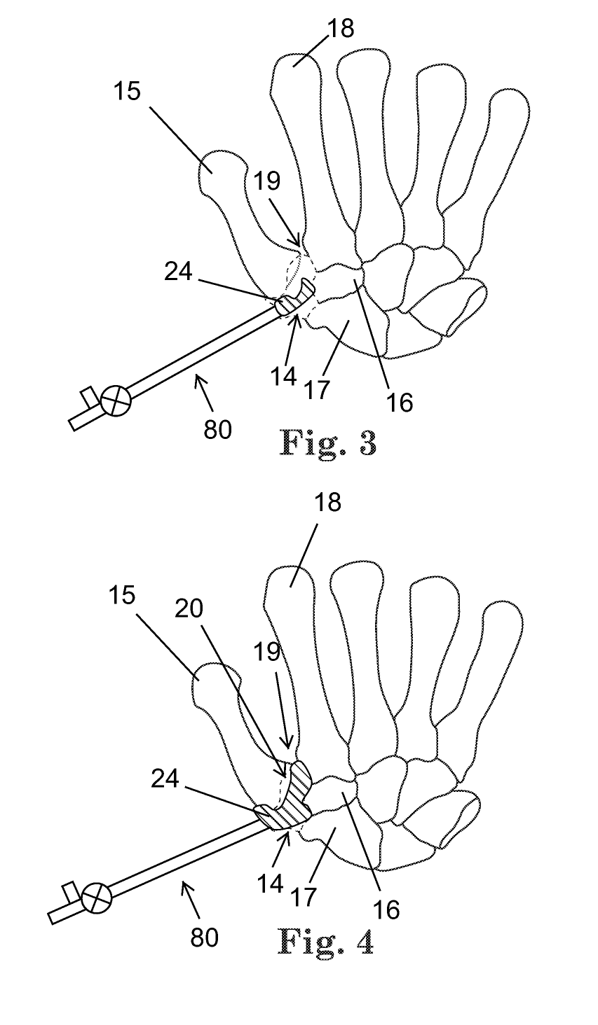

In some embodiments, the carpometacarpal (CMC) implant 20 is configured for implantation in the trapezial space 14 to restore and maintain the trapezial space 14 and stabilize subluxation of the CMC joint 10. The implant 20 optionally includes a body 22 and an optional outer cover 24 (designated by broken lines in FIG. 2) surrounding the body 22, although in some embodiments the implant 20 is a single, monolithic structure. As subsequently described, in some embodiments the implant 20 is preformed (also described as a fixed or preconfigured structure) to a final configuration and in other embodiments the implant 20 is adapted to be inflated or expanded from an initial configuration (FIG. 3) to a second, final configuration (FIG. 4).

The implant 20 generally exhibits substantial mechanical stiffness and compressive strength while being shaped to allow for mobility and stability of the CMC joint 10. The stiffness and other mechanical properties of the implant 20 are customizable and are based on the material properties of the implant 20. In some embodiments, material forming the implant 20 provides absorption characteristics and is biologically adapted to induce tissue in-growth and vascularization to develop a stable pseudoarthrosis, for example. Material forming the implant 20 is optionally a single, homogenous or non-homogenous substance or multiple homogenous or non-homogenous substances, including one or more compounds, mixtures, layers, components, or other constituent parts, for example.

In some embodiments, the implant 20 has sufficient structural stability to withstand loading at the CMC joint 10 and maintain, or encourage a more natural position of the first metacarpal 15. The implant 20 is capable of supporting high joint compressive forces and/or shear forces encountered during typical loading of the CMC joint 10. In some embodiments, the body 22 of the implant 20 is formed of a resilient material and the cover 24 of the implant 20 is formed of a mesh and/or textile weave (e.g., a polypropylene weave jacket) structure that facilitates tissue ingrowth/incorporation into the implant 20. If desired, the body 22 and/or cover 24 of the implant 20 are bioabsorbable, being formed from poly-3-hydroxybutyrate (P3HB), poly-4-hydroxybutyrate (P4HB), tricalcium phosphate (TCP), Hydroxylapatite (HA), combinations thereof and others. In other embodiments, it is contemplated that the body 22 or portions thereof are permanently implanted and/or are non-absorbable.

In some embodiments, the implant 20 is shaped to generally mimic the form of the trapezium 12, or portions thereof. For example, the implant 20 optionally defines a first metacarpal projection 32 and a second metacarpal projection 34 spaced from the first metacarpal projection 32 to form a C-type, or cuff-type receptacle 36 for receiving the first metacarpal 15 such that the first and second metacarpal projections 32, 34 extend along either side of the first metacarpal 15. The receptacle 36 optionally acts as a support surface 36A for the first metacarpal 15 during articulation thereof. In some embodiments, the first metacarpal projection 32 is configured to extend adjacent the end portion of the first metacarpal 15 that is adjacent the CMC joint 10 and the second metacarpal projection 34 is configured to extend into the space 19 between the end portions of the first and second metacarpals 15, 18, thereby helping maintain the intermetacarpal spacing between the first and second metacarpals 15, 18.

In some embodiments, the implant 20 also includes an inward, lateral projection 38 that is spaced from the second metacarpal projection 34 to define a lateral receptacle 40 for receiving a portion of the trapezoid 16, the receptacle 40 optionally defining a support surface 40A with the trapezoid 16. The implant 20 also includes a hub portion 42, from which the projections 32, 34, 38 extend, where the lateral projection 38 and the hub portion 42 abut the scaphoid 17, serving as a support surface 42A for the scaphoid 17. The first metacarpal projection 32 and the inward, lateral projection 38 optionally extend at an angle from one another of from about 105 to about 165 degrees, for example.

As shown in FIGS. 3 and 4, and as previously referenced, in some embodiments the implant 20 is adapted to be inflatable, or expandable, such that the implant 20 is implantable in a first, compact state as shown in FIG. 3 and then caused to transition to a second, inflated or expanded state that is of substantially greater size that the first, compact state as shown in FIG. 4. In some embodiments, once inflated the implant 20 provides structure adapted for tissue incorporation and absorption of the implant 20. The inflatable shape of the bioabsorbable implant 20, according to some embodiments, or permanent implant 20, according to other embodiments, allows custom conformity to the joint space of the patient and stabilization of the joint by locking onto and between joint spaces.

FIG. 3 shows the cover 24 of the implant 20 attached to a delivery device 80 for injecting material forming the body 22 of the implant 20 into the cover 24. Because the implant 20 is inflatable, the implant 20 is optionally delivered through various minimally invasive surgical techniques. For example, in some embodiments the implant 20 is inflatable such that the implant 20 is configured to be delivered into the trapezial space 14 through a minimally invasive surgical approach, such as a percutaneous incision, arthroscopically, or a minimally invasive open incision procedure.

In some embodiments, the delivery device 80 is optionally substantially similar to a catheter or needle structure that is configured to be percutaneously inserted into the hand to deliver the cover 24 into the trapezial space 14. The delivery device 80 and/or the cover 24 optionally includes radiopaque marking or other means for visualizing a percutaneous location of the device 80 and/or cover 24 during delivery of the cover 24 into the trapezial space 14. As shown, the cover 24 is releasably attached to a distal end of the device 80, for example using releasable sutures and/or sutures that are able to be later cut using a percutaneous suture cutter, for example. Once the cover 24 is positioned as desired, an injectable material is flowed through one or more internal lumens of the delivery device 80 and into the cover 24 to form the body 22 (FIG. 2). In some embodiments, the material is injected in fluid form, in solid form (e.g., solids mixed with fluid and/or gas), and/or in gas form.

In some embodiments, the injectable material includes two or more injectable components mixed in the device during injection and/or in the cover 24. The injectable material reacts exothermically in some embodiments, where the set time and exothermic reaction of the injectable material facilitate positioning and anchoring of the implant 20 in a desired location. For example, the injectable material optionally fills the cover 24, forming the implant 20 about the first metacarpal 15 and/or adjacent carpals, such as the trapezoid 16 and/or scaphoid 17, thereby positioning and anchoring the implant 20 in the trapezial space 14. The injectable material optionally sets once it is injected into the implant 20, where the injectable material is optionally provided in the form of microbeads, self-setting cement, hydrogels, combinations thereof, and other materials that set once injected into the cover 24.

The cover 24 is optionally configured to cause the implant 20 to take the desired, predetermined shape of the implant 20 upon filling the cover 24 with the injectable material. In other words, the cover 24 optionally constrains the injectable material to the desired shape. In other embodiments, the cover 24 is configured to allow the implant 20 to fill in the trapezial space 14 and conform to the adjacent boney structures. In still other embodiments, a combined approach it utilized where the cover 24 is configured such that certain portions (e.g., the first and second metacarpal projections 32, 34) take on a pre-determined shape and other portions (e.g., the hub 42) take on the shape defined by the trapezial space 14. If desired, the injection process is also optionally used to pressurize the cover 24 in order to force open, or otherwise enlarge the trapezial space 14 during injection—in other words the space is enlarged by injecting the injectable material until a desired spacing is achieved using expansion of the implant 20.

In some embodiments, the injectable material is solid (i.e., is formed without substantial voids after the material has set). In other embodiments, the injectable material has a foamy or porous structure upon setting, including honeycomb structures, for example. The shape and size of the pores on the implant are optionally selected to encourage tissue in-growth and revascularization, to develop a stable pseudoarthrosis, for example. In some embodiments, the foamy structure is formed by adding gas to the injectable material, such as nitrogen, during injection, by injecting the body material as a two part mixture that chemically reacts to form the porous structure, and/or by injecting material that includes porous components (e.g., an injectable polymer carrying porous bone matter or the like).

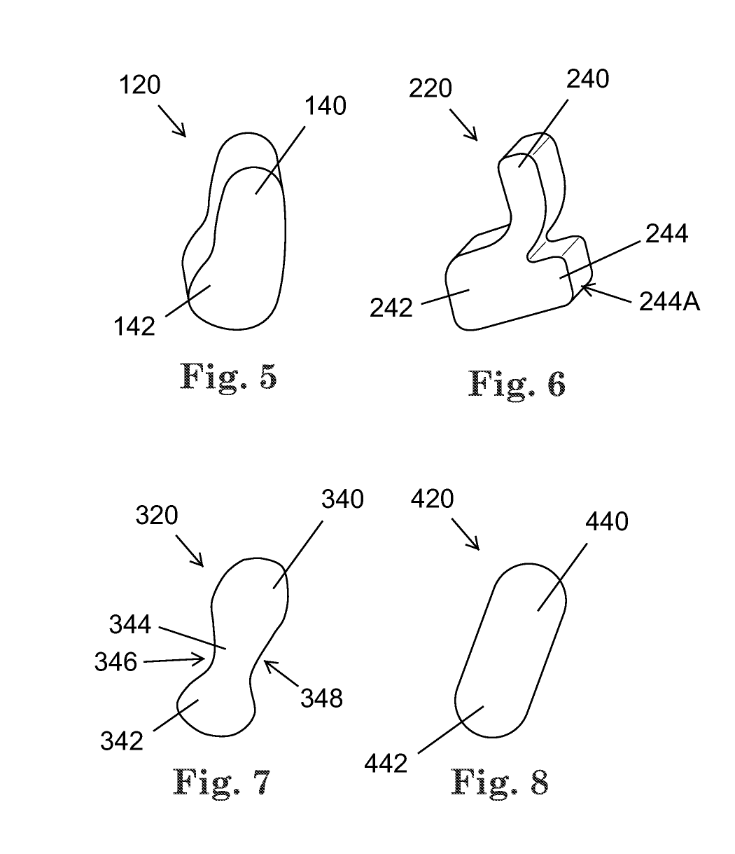

Though the implant 20 is described and shown in FIGS. 1-4 having a shape considered appropriate for some implementations, it should be understood that a variety of alternatively shaped implants are also contemplated. For example, FIGS. 5-8 show additional CMC implants 120, 220, 320, 420, according to other embodiments. Generally, the implants 120, 220, 320, 420 are able to be formed of similar materials and/or using similar methods to the implant 20.

FIG. 5 shows the implant 120 which includes an intermetacarpal projection 140 extending in a first direction and a lateral bumper 142 extending in a second direction that is substantially orthogonal to the first direction. The implant 120 generally defines a shape similar to the letter “d,” wherein the bumper 142 optionally acts to support the first metacarpal 15 and the intermetacarpal projection 140 acts to maintain spacing between the first and second metacarpals 15, 18, according to some embodiments.

FIG. 6 shows the implant 220 which includes an intermetacarpal projection 240, a lateral bumper 242, and an inner lateral projection 244. As shown, the intermetacarpal projection 240 and the inner lateral projection 244 define a receptacle which, according to some embodiments, forms a support surface 244A for receiving a portion of the trapezoid 16 and also provides for some flex between projections 240, 244 (e.g., during articulation of the first and/or second metacarpals 15, 18. The intermetacarpal projection 240 is optionally adapted to fit in the space 19 to help maintain spacing between the first and second metacarpals 15, 18. The lateral bumper 242 is optionally of sufficient dimensions to help maintain position of the first metacarpal 15 under axial loading, for example.

FIG. 7 shows the implant 320 which has a biconcave shape (e.g., analogous to that of a red blood cell). The implant 320 includes a first lobe 340, also described as a metacarpal projection, a second lobe 342, also described as a metacarpal projection, and a central portion 344 between the first and second lobes 340, 342, the central portion 344 defining a metacarpal receptacle 346 and a trapezoid-scaphoid receptacle 348 generally opposite the metacarpal receptacle. The first lobe 340 optionally assists with maintaining intermetacarpal spacing between the first and second metacarpals 15, 18 while the metacarpal receptacle 346 is configured to receive the first metacarpal 15 and promote effective articulation thereof. The trapezoid-scaphoid receptacle 348 interacts with the carpal bones adjacent the trapezial space 14 to help maintain a position of the implant 320 in the space 14.

FIG. 8 shows the implant 420 which is optionally shaped as a prolate spheroid, also described as a “jelly bean” shape. The implant 420 is sized and shaped, or otherwise configured to be inserted into the trapezial space 14 to help maintain appropriate positioning and articulation of the first metacarpal 15 as well as proper spacing between the first and second metacarpals 15, 18. The implant 420 includes a first end portion 440 and a second end portion 442 that each are substantially rounded in shape.

Various modifications, permutations, and additions can be made to the exemplary embodiments and aspects of the embodiments discussed without departing from the scope of the present invention. For example, while the embodiments described above refer to particular features, the scope of this invention also includes embodiments having different combinations of features and embodiments that do not include all of the features. Accordingly, the scope of the present invention is intended to embrace all such alternatives, modifications, permutations, and variations as fall within the scope of the claims, together with all equivalents thereof.

1. An implant for a joint including a first bone having a first end portion and a second bone having a second end portion, the first and second end portions extending next to one another and being spaced from one another, the implant comprising:
a body formed of resilient material, the body having:
a first projection adapted to extend into a space between the first and second end portions of the first and second bones; and
a receptacle for receiving the first end portion of the first bone.

2. The implant of claim 1, wherein the implant is adapted for insertion into a trapezial space left by excision of a trapezium from a carpometacarpal (CMC) joint, the first projection being a first metacarpal projection adapted to extend into an intermetacarpal space between the first metacarpal of a hand and a second metacarpal of the hand that is adjacent to the first metacarpal.

3. The implant of claim 2, wherein the receptacle is adapted to receive the first metacarpal.

4. The implant of claim 2, wherein the body further comprises an inward, lateral projection that is spaced from the second metacarpal projection to define a lateral receptacle for receiving a portion of a trapezoid bone of the hand.

5. The implant of claim 4, wherein the body further comprises a hub from which the first and second metacarpal projections and the lateral projection extend.

6. The implant of claim 1, further comprising a cover formed of a mesh material, the cover surrounding the body.

7. The implant of claim 1, wherein the body includes a porous structure.

8. The implant of claim 1, wherein the body is formed of bioabsorbable material.

9. The implant of claim 1, wherein the body is formed of material selected from a group consisting of: poly-3-hydroxybutyrate (P3HB), poly-4-hydroxybutyrate (P4HB), tricalcium phosphate (TCP), Hydroxylapatite (HA), and combinations thereof.

10. The implant of claim 1, wherein the body is formed of material selected from a group consisting of: microbeads, self-setting cement, hydrogels, and combinations thereof.

11. A method of delivering an implant into a joint following excision of a bone from the joint, the method comprising:
excising at least a portion of a bone from a joint to leave a bone space, where a first end portion of a first bone extends next to a second end portion of a second bone, the first end portion of the first bone being spaced apart from the second end portion of the second bone;
accessing the bone space with a delivery device maintaining a cover of an implant, the cover being collapsed such that the implant is in a first, substantially compact state;
positioning the cover of the implant at a desired location in the bone space;
injecting material through the delivery device into the cover to transition the implant to a second, expanded state that is of substantially greater size that the first, compact state, such that at least a portion of the implant extends into the space between the first and second end portions of the first and second bones, respectively; and
allowing the injectable material to set to form a substantially resilient body of the implant.

12. The method of claim 11, wherein the bone space is percutaneously accessed with the delivery device and the cover is positioned percutaneously with the delivery device.

13. The method of claim 11, wherein the bone space is a trapezial space and the first and second bones are metacarpal bones.

14. The method of claim 11, wherein transitioning the implant to the second, expanded state that is of substantially greater size that the first, compact state includes forming first and second metacarpal projections of the implant, the first and second metacarpal projections extending on either side of a first metacarpal of the hand.

15. The method of claim 11, wherein injecting the material includes anchoring the implant to a first metacarpal of the hand.

16. The method of claim 11, wherein the material sets as part of an exothermic reaction.

17. The method of claim 11, wherein after setting the body of the implant is substantially porous.

18. The method of claim 11, wherein the material comprises at least one of poly-3-hydroxybutyrate (P3HB), poly-4-hydroxybutyrate (P4HB), and tricalcium phosphate (TCP), Hydroxylapatite (HA).

19. The method of claim 11, wherein the material comprises a plurality of components that are mixed together as part of injecting the material into the cover.

20. The method of claim 11, wherein the cover constrains the material to a predefined shape of the implant.

21. The method of claim 11, wherein injecting the material includes enlarging the bone space.

22. The method of claim 11, wherein the material comprises at least one of microbeads, self-setting cement, and hydrogels.

23. The method of claim 11, wherein the material is bioabsorbable.
